SEQUENCE LISTING (4 sequences)

SEQ ID NO: 1 - protein, 6 aa, Ganoderma microsporum
LAWNVK

SEQ ID NO: 2 - protein, 10 aa, Ganoderma microsporum
DLGVRPSYAV

SEQ ID NO: 3 - protein, 111 aa, Ganoderma microsporum
MSDTALIFTLAWNVKQLAFDYTPNWGRGRPSSFIDTVTFPTVLTDKAYTYRVVVSGKDLGVRPSYAVESDGSQKINFLEYNSGYGIADTNTIQVYVIDPDTGNNFIVAQWN

SEQ ID NO: 4 - protein, 140 aa, Ganoderma microsporum
EAEAEFMSDTALIFTLAWNVKQLAFDYTPNWGRGRPSSFIDTVTFPTVLTDKAYTYRVVVSGKDLGVRPSYAVESDGSQKINFLEYNSGYGIADTNTIQVYVIDPDTGNNFIVAQWNYLEQKLISEEDLNSAVDHHHHHH

(19) United States
(12) Patent Application Publication
(10) Pub. No.: US 2023/0233632 A1
(43) Pub. Date: Jul. 27, 2023

(54) IMMUNOMODULATORY PROTEIN FOR PREVENTION OF CORONAVIRUS INFECTION

(71) Applicant: MYCOMAGIC BIOTECHNOLOGY CO., LTD, New Taipei City (TW)

(72) Inventors: Tung-Yi LIN, Taipei (TW); Ming-Han TSAI, Taipei (TW)

(21) Appl. No.: 17/821,291

(22) Filed: Aug. 22, 2022

Related U.S. Application Data
(60) Provisional application No. 63/266,712, filed on Jan. 12, 2022.

(51) Int. Cl. A61K 36/074; A61K 38/16; A61P 31/14

(52) U.S. Cl. CPC A61K 36/074; A61K 38/16; A61P 31/14

(57) ABSTRACT

The present disclosure pertains to the use of an immunomodulatory protein in preventing, inhibiting and/or reducing coronavirus (CoV) infections. The Ganoderma immunomodulatory protein, a recombinant thereof or a fragment thereof of the disclosure is able to induce ACE2 degradation via activating the protein degradation system.

### PRIORITY INFORMATION

This application claims the benefit of priority under 35 U.S.C. 119(e) to U.S. Provisional Patent Application No. 63/266,712, filed Jan. 12, 2022, which is incorporated herein by reference in its entirety.

### SEQUENCE LISTING

The instant application contains a Sequence Listing which has been filed electronically in XML format and is hereby incorporated by reference in its entirety. Said XML copy, created on Apr. 17, 2023 is named “G4590-15500US_SeqListing_20230417.xml” and is 5 kilobytes in size.

### FIELD OF THE INVENTION

The present disclosure relates to the field of prevention and/or inhibition and/or reduction and/or elimination of coronavirus infections and/or likelihood of coronavirus infections. Particularly, the present disclosure pertains to the use of an immunomodulatory protein in preventing, inhibiting, reducing and/or eliminating coronavirus (CoV) infections and/or likelihood of coronavirus infections.

### BACKGROUND OF THE INVENTION

Coronavirus disease-19 (COVID-19), which is caused by severe acute respiratory syndrome coronavirus 2 (SARS-CoV-2), is still a global pandemic. Currently, there are already vaccines that can be used to prevent this viral infection; however, the rate of vaccine delivery is not universal among all countries. In addition, some reports show that SARS-CoV-2 may break through the vaccine protection and still cause a risk of infection [References 1-3]. Therefore, in addition to the development of vaccines, the use of other strategies to improve vaccine efficiency or reduce viral infections is an urgent and important concern.

It is well-known that Spike (S) protein of SARS-CoV-2 is a major protein which specifically recognizes and binds to the angiotensin converting enzyme 2 (ACE2) of host cells, resulting in infection [References 4, 5]. Therefore, there has been an increase in the number of studies that focus on how to block SARS-CoV-2-S/ACE2 interactions, which could become a potential therapeutic means of preventing infection. Currently, recombinant ACE2 proteins, ACE2-neutralizing antibodies, engineered ACE2 traps, etc., have been investigated to exploit their potential efficacy of interfering with SARS-CoV-2-S/ACE2 interactions [References 6-10].

However, there is still a need to develop a new strategy against coronavirus infection.

### SUMMARY OF THE INVENTION

The present disclosure is based in part on the preventive measures of CoV infection. It was surprisingly found that an immunomodulatory protein from Ganoderma can prevent, inhibit, reduce and/or eliminate coronavirus infection and/or likelihood of coronavirus infections, such as Severe Acute Respiratory Syndrome Coronavirus Type 2 (SARS-CoV-2).

In one aspect, the present disclosure provides a method for preventing, inhibiting, reducing and/or eliminating CoV infection and/or likelihood of CoV infection in a subject, comprising administering an effective amount of Ganoderma immunomodulatory protein, a recombinant thereof or a fragment thereof to the subject. Alternatively, the present disclosure provides Ganoderma immunomodulatory protein, a recombinant thereof or a fragment thereof for use in a method for preventing, inhibiting, reducing and/or eliminating CoV infection and/or likelihood of CoV infection in a subject.

In another aspect, the present disclosure provides a method for preventing, inhibiting, reducing and/or eliminating CoV infection and/or likelihood of CoV infection in a subject, comprising administering a composition comprising an effective amount of Ganoderma immunomodulatory protein, a recombinant thereof or a fragment thereof to the subject. Alternatively, the present disclosure provides a composition comprising an effective amount of Ganoderma immunomodulatory protein, a recombinant thereof or a fragment thereof for use in a method for preventing, inhibiting, reducing and/or eliminating CoV infection and/or likelihood of CoV infection in a subject.

In one embodiment, the Ganoderma immunomodulatory protein, a recombinant thereof or a fragment thereof is derived from Ganoderma lucidum, Ganoderma tsugae, Ganoderma microsporum or Ganoderma sinensis. In a further embodiment, the Ganoderma immunomodulatory protein, a recombinant thereof or a fragment thereof is derived from Ganoderma microsporum.

In one embodiment, the Ganoderma immunomodulatory protein or a recombinant thereof described herein comprises an amino acid sequence of SEQ ID NO: 3. In one embodiment, the recombinant of Ganoderma immunomodulatory protein comprises an amino acid sequence of SEQ ID NO: 4. In one embodiment, the fragment of Ganoderma immunomodulatory protein comprises an amino acid sequence selected from the group consisting of SEQ ID NOs: 1 to 2.

The sequences of SEQ ID NO: 1 to 4 are listed as follows.

(SEQ ID NO: 1)
LAWNVK
(SEQ ID NO: 2)
DLGVRPSYAV
(SEQ ID NO: 3)
MSDTALIFTLAWNVKQLAFDYTPNWGRGRPSSFIDTVTFPTVLTDKA
YTYRVVVSGKDLGVRPSYAVESDGSQKINFLEYNSGYGIADTNTIQV
YVIDPDTGNNFIVAQWN
(SEQ ID NO: 4)
EAEAEFMSDTALIFTLAWNVKQLAFDYTPNWGRGRPSSFIDTVTFPT
VLTDKAYTYRVVVSGKDLGVRPSYAVESDGSQKINFLEYNSGYGIAD
TNTIQVYVIDPDTGNNFIVAQWNYLEQKLISEEDLNSAVDHHHHHH

In some embodiments of the disclosure, the CoV described herein is alpha-CoV, beta-CoV, gamma-CoV, and delta-CoV2. In some embodiments, the CoV described herein includes, but is not limited to, SARS-CoV, MERS-CoV or SARS-CoV-2.

In one embodiment, the Ganoderma immunomodulatory protein or a recombinant thereof described herein induces ACE2 degradation. In a further embodiment, the ACE2 degradation is induced via activating the protein degradation system, including proteasome and lysosome.

In one embodiment of the disclosure, the subject is vaccinated or non-vaccinated. In one embodiment, the subject is vaccinated with a coronavirus vaccine. In some embodiments, the coronavirus vaccine is a coronavirus mRNA vaccine, a coronavirus viral vector vaccine or a coronavirus spike protein vaccine. In some embodiments, the coronavirus vaccine is COVID-19 coronavirus vaccine.

In one embodiment, the subject is treated or has been treated with a therapeutic agent against CoV.

In some embodiments of the disclosure, the subject is administered one or more further therapeutic agents against CoV before or after or at the same time the Ganoderma immunomodulatory protein or a recombinant thereof or a fragment is administered.

In some embodiments of the disclosure, the Ganoderma immunomodulatory protein or a recombinant thereof or a fragment can be administered with one or more further therapeutic agents against CoV concurrently, sequentially, or separately.

In some embodiments of the disclosure, the Ganoderma immunomodulatory protein or a recombinant thereof or a fragment is administered orally, parenterally, topically or by inhalation.

In some embodiments of the disclosure, the Ganoderma immunomodulatory protein or a recombinant thereof or a fragment is administered by inhaler to the respiratory tract.

### BRIEF DESCRIPTION OF THE DRAWINGS

The subject patent or application file contains at least one drawing executed in color. Copies of this patent or patent application publication with color drawing(s) will be provided by the Office upon request and payment of the necessary fee.

FIGS. 1 A to D. GMI did not inhibit cell viability of HEK293T/ACE2 cells but inhibited SARS-CoV-2 pseudovirus infection. (A) HEK293T/ACE2 cells were treated with various concentrations of GMI (0-1.2 μM) for 24-72 h. The viability of HEK293T/ACE2 cells was evaluated using MTT assay. (B) Scheme for HEK293T/ACE2 cells were treated with GMI and SARS-CoV-2 pseudovirus. (C-D) HEK293T/ACE2 cells were co-treated with various concentrations of GMI (0-0.6 μM) and infected with SARS-CoV-2 pseudovirus for 48 h. The infected cells were GFP-positive observed by fluorescence microscopy (C) and the percentage of GFP-positive cells in each sample was quantified by flow cytometry (D). Each GMI-treated group was normalized by the control (no-treatment) group (D). The data were representative of more than three separate experiments and were presented as mean SDs; the error bars indicated SD. Significant differences were noted (***P<0.001).

FIGS. 2 A to D. GMI interacted with S2 domain of SARS-CoV-2-S to interfere with the interaction between ACE2 and SARS-CoV-2-S. (A) Schematic design of the in vitro GMI (0.3 μM)-treated SARS-CoV-2 pseudovirus infection experiment. (B-C) SARS-CoV-2 pseudovirus was pretreated with GMI for 10 mins and pelleted down by concentration to remove the excess GMI from viral supernatant. The viruses were therefore resuspended in fresh culture medium and then added to HEK293T/ACE2 cells for another 48 h. The infected cells were GFP positive observed by fluorescence microscopy (B) and the percentage of GFP-positive cells in each sample was quantified by flow cytometry (C). The data were representative of three separate experiments and were presented as mean SDs; the error bars indicated SD. Significant differences were noted (EXP vs CTL, *P<0.05). (D) Up panel: different constructs of SARS-CoV-2-S. Down panel: representative immunoblots of HEK293T/ACE2 cells transformed with different mutant constructs of SARS-CoV-2-S and incubated with GMI (0.3 μM) for 1 h.

FIGS. 3 A to F. GMI-induced endocytosis and degradation of ACE2 in HEK293T/ACE2 cells. (A) HEK293T/ACE2 cells were treated with various concentrations of GMI (0-0.6 μM) for 3, 24 and 48 h. (B) ACE2 downregulation was shown depending on time course after the incubation of cycloheximide (CHX; 100 μg/ml) in the presence or absence of GMI (0.3 μM) for 0-24 h in 293T-ACE2 cells. Quantification of the intensities of the bands of ACE2 were representatives of three independent determinations by ImageJ. Relative ACE2 levels in each sample were quantified by densitometry as a function of time. (C-D) HEK293T/ACE2 cells were pretreated with DMSO (vehicle control) or MG132 (10 μM; C)/Bafilomycin A1 (BafA1; 20 μM; D) for 30 min, followed by incubation with GMI (0.6 μM) for 3 and 24 h. (E-F) HEK293T/ACE2 cells were pretreated with DMSO (vehicle control) or dynasore (200 μM; E)/MβCD (20 mM; F) for 30 min, followed by incubation with GMI (0.6 μM) for 3 and 24 h. The expressions of ACE2 protein were determined by Western blot. Tubulin was used as internal control. Data were presented as the mean±SD; error bars indicated SDs. Significant differences were shown (*P<0.05 compared to the control group).

FIGS. 4 A to E. Discontinuous GMI exposure preserved inhibition of the SARS-CoV-2 pseudovirus infection. (A) HEK293T/ACE2 cells were treated with various concentrations of GMI (0-1.2 μM) for 48 h, and then GMI was removed for 24-48 h to analyze the cell viability of HEK293T/ACE2 cells. The cell viability was evaluated using MTT assay. (B) HEK293T/ACE2 cells were treated with GMI (0.6 μM) for 48 h. GMI was then removed from cells to analyze the ACE2 levels in another 24 h and 48 h. The expressions of ACE2 protein were determined by Western blot. Tubulin was used as internal control. The intensities of the bands of ACE2 were quantified by ImageJ. Data were presented as the mean±SD; error bars indicated SDs. Significant differences were shown (***P<0.001 compared to the control (CTL) group; ^{#}P<0.05 compared to GMI treatment for 48 h). (C) The schematic design was shown for the continuous and discontinuous GMI treatment in inhibition of the SARS-CoV-2 pseudovirus infection. HEK293T/ACE2 cells were pretreated with GMI (0.3 μM) for 48 h and then the cells were infected with SARS-CoV-2 pseudovirus for 48 h under continuous GMI treatment or not. The infected cells were GFP positive observed by fluorescence microscopy (E) and the percentage of GFP-positive cells in each sample was quantified by flow cytometry (D). Data were presented as the mean±SD; error bars indicated SDs. Significant differences were shown (***P<0.001 compared to the control (CTL) group; ^{#}P<0.05 compared to each discontinuous GMI treatment group).

FIGS. 5 A to F. GMI-induced endocytosis and degradation of ACE2 in lung WI38-2RA cells. (A) WI38-2RA cells were treated with various concentrations of GMI (0-1.2 μM) for 48 and 72 h. The viability of WI38-2RA cells was evaluated using MTT assay. Each GMI-treated group was normalized by the control group. (B) WI38-2RA cells treated with GMI (0-0.6 μM) for 3 and 24 h. (C-F) WI38-2RA cells were pretreated with DMSO (vehicle control) or MG132 (10 μM; C)/Bafilomycin A1 (BafA1; 20 μM; D)/dynasore (200 μM; E)/MβCD (20 mM; F) for 30 mins, followed by incubation with GMI (0.6 μM) for 24 h. The expressions of ACE2 protein were determined by Western blot. Tubulin was used as internal control. The data were representative of more than three separate experiments and were presented as mean SDs; the error bars indicated SD. Significant differences were noted (*P<0.001).

FIG. 6. SARS-CoV-2 pseudovirus infects with HEK293T/ACE2 cells. HEK293T/ACE2 or HEK293T cells were infected with SARS-CoV-2 pseudovirus for 48 h. The infected cells were GFP-positive observed by fluorescence microscopy.

FIGS. 7 A and B. GMI slightly blocks the interaction between SARS-CoV-2-S and ACE2. (A) ACE2 proteins were pre-incubated with GMI (0, 0.6 and 1.2 μM) for 1 h. After the incubation, the solution was added into the spike protein-coating well for 2.5 h to detect the interaction between ACE2 and spike. (B) GMI (0, 0.6 and 1.2 μM) was added to the spike protein-coating well for 1 h. After the incubation, ACE2 protein was added into the well for 2.5 h to detect the interaction between ACE2 and spike. Data were presented as the mean±SD; error bars indicated SDs. Significant differences were shown (*P<0.05 compared to the untreated (control) group)).

FIG. 8. GMI does not inhibit the VSV-G infection. HEK293T cells were co-treated with PBS (CTL) or GMI at 0.3 μM and infected with VSV-G pseudovirus carrying a GFP gene driven by CMV promoter for 48 h. The infected cells were GFP-positive visualized by using a fluorescence microscope. Right panel: The bar figure shows the percentage of GFP-positive cells in each sample quantified by flow cytometry.

### DETAILED DESCRIPTION OF THE INVENTION

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described. All publications mentioned herein are incorporated for reference.

In this application, the use of the singular includes the plural, the article “a” or “an” means “at least one,” and the use of “or” means “and/or,” unless specifically stated otherwise.

The term “preventing” or “prevention” is recognized in the art, and when used in relation to a condition, it includes administering an agent to reduce the frequency or severity of or to delay the onset of symptoms of a medical condition in a subject, relative to a subject which does not receive the agent.

The terms “Ganoderma immunomodulatory protein” and “GMI” is interchangeably used herein.

As interchangeably used herein, the terms “individual,” “subject,” “host,” and “patient,” refer to a mammal, including, but not limited to, murines (rats, mice), non-human primates, humans, canines, felines, ungulates (e.g., equines, bovines, ovines, porcines, caprines), etc.

As used herein, the term “prevent” refers to any success or indicia of success in the forestalling of CoV infection in subjects in clinical remission.

As used herein, the term “effective amount” means an amount effective to achieve a particular biological result such as, but not limited to, biological results disclosed, described, or exemplified herein. Such results can include, but are not limited to, the prevention of CoV infection.

As used herein, “subject” refers to either a human or non-human animal.

The term “coronavirus” or “CoV” refers to any virus of the coronavirus family, including, but not limited to, SARS-CoV-2, MERS-CoV, and SARS-CoV SARS-CoV-2 refers to the newly-emerged coronavirus which is rapidly spreading to other areas of the globe. It binds via the viral spike protein to human host cell receptor angiotensin-converting enzyme 2 (ACE2). The spike protein also binds to and is cleaved by TMPRSS2, which activates the spike protein for membrane fusion of the virus.

The term “coronavirus infection” or “CoV infection,” as used herein, refers to infection with a coronavirus such as SARS-CoV-2, MERS-CoV, or SARS-CoV. The term includes coronavirus respiratory tract infections, often in the lower respiratory tract. Symptoms can include high fever, dry cough, shortness of breath, pneumonia, gastro-intestinal symptoms such as diarrhea, organ failure (kidney failure and renal dysfunction), septic shock, and death in severe cases.

The present disclosure surprising found that the Ganoderma immunomodulatory protein, a recombinant thereof or a fragment thereof is able to induce ACE2 degradation via activating the protein degradation system, including proteasome and lysosome. In addition, the Ganoderma immunomodulatory protein, a recombinant thereof or a fragment thereof triggers dynamin and lipid raft-mediated ACE2 endocytosis.

Accordingly, the present disclosure provides a method for preventing, inhibiting, reducing and/or eliminating CoV infection and/or likelihood of CoV infection in a subject, comprising administering an effective amount of Ganoderma immunomodulatory protein, a recombinant thereof or a fragment thereof to the subject.

Fungal immunomodulatory proteins (FIPs) are a group of proteins found in fungi, which are extensively studied for their immunomodulatory activity including activation of immune cells, leading to immune-mediated anti-allergic, anti-inflammatory, and anti-tumor effects. Ganoderma immunomodulatory protein is one kind of FIP derived from Ganoderma.

GMI is a kind of fungal immunomodulatory protein (FIP) from Ganoderma microsporum. In the past ten years, GMI has been proven to have multifaceted anti-cancer activities, especially in lung cancer. For example, GMI inhibits EGFR-mediated cell motility of lung cancer cells [Reference 12]. Meanwhile, GMI induces autophagic cell death of various lung cancer cells in vitro and in vivo via p53 and mTOR pathways [Reference 13-15]. Moreover, GMI triggers protein degradation pathways, resulting in the inhibition of lung cancer cell survival. Hsin et al. demonstrated that GMI triggers apoptosis in lung cancer cells via induction of proteasome-dependent β-catenin degradation [Reference 16]. GMI inhibits cell viability of pemetrexed-resistant lung cancer cells via induction of autophagic CD133 degradation [Reference 17].

The preparation of the Ganoderma immunomodulatory protein or the recombinant or fragment thereof has been described in U.S. Pat. No. 7,601,808. Particularly, the Ganoderma immunomodulatory protein is referred to as GMI; the recombinant of Ganoderma immunomodulatory protein is referred to as reGMI; and the fragment of Ganoderma immunomodulatory protein is referred to as SEQ ID NOs: 2 and 3 in U.S. Pat. No. 7,601,808.

In one embodiment of the present disclosure, the fungal protein, GMI, is use to prevent, inhibit, reduce and/or eliminate SARS-CoV-2 infection. The present disclosure firstly provides the evidence indicating that GMI effectively inhibits SARS-CoV-2-S pseudovirus infection. Moreover, it is found that GMI could bind to S2 domain of SARS-CoV-2-S and slightly interfere with the interaction between spike and ACE2. Importantly, the present disclosure demonstrates that GMI downregulated ACE2 levels on the host cells. Specifically, GMI promotes clathrin and lipid raft-dependent ACE2 endocytosis, resulting in induction of ACE2 degradation. These significant results suggest that GMI would be a promising prevention agent to alleviate SARS-CoV-2 infection.

The present disclosure develops a SARS-CoV-2 spike lentiviral pseudovirus encoding a green fluorescent protein (GFP) gene, and demonstrates that GMI inhibits GFP expressing SARS-CoV-2 pseudovirus from infecting ACE2 overexpressing HEK293T (HEK293T/ACE2) cells. In parallel, GMI reduces ACE2 expression in HEK293T/ACE2 and lung WI38-2RA cells. Mechanistically, GMI induces ACE2 degradation via activating the protein degradation system, including proteasome and lysosome. Abolishing proteasome and lysosome by MG132 and bafilomycin A1, respectively, rescues GMI-reduced ACE2 levels. In addition, GMI triggers dynamin and lipid raft-mediated ACE2 endocytosis. In conclusion, GMI prevents SARS-CoV-2 pseudovirus infection via induction of ACE2 degradation in host cells, suggesting that GMI will be a promising agent for the prevention of COVID-19.

The GMI or a recombination or a fragment thereof of the disclosure can be administered to a patient either alone or in pharmaceutical compositions where it is mixed with suitable carriers and excipients. The GMI or a recombination or a fragment thereof or composition of the disclosure can be administered parenterally, such as by intravenous injection or infusion, intraperitoneal injection, subcutaneous injection, or intramuscular injection. The GMI or a recombination or a fragment thereof or composition can be administered orally or rectally through appropriate formulation with carriers and excipients to form tablets, pills, capsules, liquids, gels, syrups, slurries, suspensions and the like. The immunomodulatory protein, recombination thereof or composition can be administered topically, such as by skin patch. The GMI or a recombination or a fragment thereof or composition can be formulated into topical creams, skin or mucosal patch, liquids or gels suitable to topical application to skin or mucosal membrane surfaces. The immunomodulatory protein, recombination thereof or composition can be administered by inhaler to the respiratory tract for local or systemic treatment of CoV diseases (such as Covid-19).

The subject receiving the GMI or a recombination or a fragment thereof can be non-vaccinated or vaccinated. In some embodiments, the subject is vaccinated with a coronavirus vaccine. In some embodiments, the coronavirus vaccine is a coronavirus mRNA vaccine, a coronavirus viral vector vaccine or a coronavirus protein vaccine.

The dosage of the GMI or a recombination or a fragment thereof or composition suitable for use according to the present disclosure can be determined by those skilled in the art on the basis of the disclosure herein. The medicament will contain an effective dosage (depending upon the route of administration and pharmacokinetics of the active agent) of suitable pharmaceutical carriers and excipients suitable for the particular route of administration of the formulation (i.e., oral, parenteral, topical or by inhalation). The GMI or a recombination or a fragment thereof is mixed into the pharmaceutical composition by means of mixing, dissolving, granulating, dragee-making, emulsifying, encapsulating, entrapping or lyophilizing processes. The pharmaceutical compositions for parenteral administration include aqueous solutions of the inventive polypeptide in water-soluble form. Additionally, suspensions of the inventive polypeptide may be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. The suspension may optionally contain stabilizers or agents to increase the solubility of the complex or combination to allow for more concentrated solutions.

The GMI or a recombination or a fragment thereof can be administered with, before or after one or more further therapeutic agents against CoV. The GMI or a recombination or a fragment thereof can be administered with one or more further therapeutic agents against CoV concurrently, sequentially or separately.

Examples of the therapeutic agents against CoV include, but are not limited to, nirmatrelvir, ritonavi, remdesivir, babtelovimab, molnupiravir and any combination thereof.

The following examples are provided to aid those skilled in the art in practicing the present disclosure.

### Example

### 1. Material and Methods

1.1. Materials and Chemicals

GMI, dissolved in PBS, was obtained from MycoMagic Biotechnology Co., Ltd. (New Taipei, Taiwan).

1.2. Cell Culture

HEK293T cell line is a derivative of HEK293 cells that contains the SV40 T-antigen (ATCC: CRL-3216). HEK293T cells with stable expression of human ACE2 (HEK293T/ACE2) were obtained after transduction with lentiviruses carrying hACE2 and blasticidin resistance genes, and hence were selected with 10 μg/ml blasticidin (Invitrogen). HEK-293T cell line and its derivatives were cultured in RPMI-1640 medium (Gibco) supplemented with 10% FBS and 100 U/mL penicillin and streptomycin (Biological industries, Cromwell, Conn.). The normal human lung fibroblast WI-38 VA-13 subline 2RA (WI38-2RA) cells were purchased from the Bioresource Collection and Research Center of the Food Industry Research and Development Institute (BCRC, Hsinchu, Taiwan). WI38-2RA cells were cultured in Minimum essential medium (MEM, Gibco) supplemented with 10% fetal bovine serum (FBS, HyClone), 2 mM L-glutamine, 1.5 g/L sodium bicarbonate, 0.1 mM non-essential amino acids, and 1.0 mM sodium pyruvate. All cells were cultured at 37° C. under a mixture of 95% air and 5% CO₂.

1.3. Plasmids

pLAS2 is a lentiviral vector with the CMV promoter and was obtained from the RNAi core of Academia Sinica, Taiwan. We therefore inserted a GFP gene after the CMV promoter of the pLAS2 lentiviral plasmid (pLAS2-GFP). pcDNA3.1-SARS2-Spike is an expression plasmid carrying the wild-type (Wuhan strain) SARS-CoV-2 spike protein with a C9 tag at its terminus and was a gift from Fang Li (Addgene plasmid #145032). We further modified this Spike-expressing plasmid with D614G plus N501Y (D614G/N501Y) mutations at Spike gene by PCR cloning and applied it to all this research for pseudovirus production and transfection experiments. Other spike mutations were constructed based on pcDNA3.1-SARS2-Spike D614G/N501Y plasmid through restriction enzyme digestion or overlapping PCR, which included the deletion mutations with the indicated amino acids 586-978, 820-1273, and 14-685. The sequences of the plasmids constructed by PCR cloning were further validated through Sanger sequencing. The lenti-ACE2 plasmid was a gift from Academia Sinica, Taiwan.

1.4. Pseudovirus Production

Pseudovirions were produced by the co-transfection of HEK293T cells with pCMVR8.91 plasmid, a lentiviral plasmid encoding CMV-promoter-driven GFP gene (pLAS2-GFP), and a plasmid encoding SARS-CoV-2 Spike gene (pcDNA3.1-Spike D614G/N501Y) at the ratio 6.25:6.5:1.1 using PEI transfection (PEI MAX, MW 40,000, Polysciences 24765-1. Polysciences, Pa., USA) following the manufacturer's instructions. The supernatants were collected at 72 h post transfection and passed through a 0.45-μm filter. The SARS-CoV-2 S pseudovirions were further concentrated by sucrose centrifugation method [Reference 18] and resuspended in RPMI medium supplemented with 10% FBS.

1.5. Cell Viability

Cells (5×10³ cells/well) were seeded into 96-well culture plate dishes and incubated overnight. Cells were treated with GMI (0-1.2 μM) as indicated for 24-72 h. After incubation, cell viability was assessed by MTT assay as described previously [Reference 19].

1.6. Immunoblotting Analysis

Cells were treated with various concentrations of GMI for indicated times. After that, cells were rinsed with PBS and harvested by scrapping the cells into lysis buffer [Reference 20] containing proteinase inhibitors (Sigma Chemical Co.). Whole cell lysates were harvested to determine the protein concentration by using a Bradford assay (Bio-Rad). Cell lysates (20-30 μg) were then subjected to Western blot analysis which was conducted as previously described [Reference 21]. The antibodies against ACE2, TMPRSS2 and tubulin were purchased from GeneTex (Hsinchu, Taiwan). The expression of tubulin was used as an internal control.

1.7. Pseudovirus Infection

To evaluate the transduction efficiency of Spike pseudovirus, 2,500 HEK293T/ACE2 cells were seeded in 96-well plates per well and treated with various concentrations of GMI at different time periods according to the experiment. After pre-treatment, the culture medium was removed and replenished with fresh medium containing GFP-encoding Spike pseudovirus with or without GMI as indicated in each experiment. The transduction efficiency of each sample was visualized 48 h post transduction by fluorescence microscopy (Zeiss) and the percentage of GFP-positive cells was quantified by flow cytometry (FACSCalibur).

1.8. Pull Down Assay

HEK293T cells were at first transfected with the indicated SARS-CoV-2 Spike-encoding plasmids for 72 h and then exposed to GMI (0.3 μM) at 37° C. for 1 h. Subsequently, the cells were washed with PBS three times and lysed in lysis buffer (20 mM Tris HCl pH 8, 137 mM NaCl, 1% Nonidet P-40, and 2 mM EDTA) at 4° C. under rotation at 150 rpm for 1 h. The lysate was centrifuged at 21,000×g for 10 mins, and the clarified lysates were collected and subjected to immunoprecipitation using a Dynabeads Protein G Immunoprecipitation kit (Invitrogen 10007D) following the manufacturer's instructions. We applied a rabbit polyclonal antibody against the SARS-CoV-2 Spike protein (obtained from Academia Sinica, Taiwan) and a mouse monoclonal antibody against the His tagged-GMI (Clone HIS.H8, ThermoFisher MA1-21315) for immunoprecipitation. After the incubation of lysates with antibody-loaded Protein G Dynabeads at room temperature for 1 h, the beads together with the pulled-down proteins were collected using a magnet and washed four times with washing buffer. The immunoprecipitates together with Dynabeads Protein G were then eluted in Laemmli buffer for 10 mins at 95° C. and separated on SDS-polyacrylamide gels for Western blotting.

1.9. Statistical Analysis

GraphPad Prism8 was used for statistical analysis. The experiments were conducted three times or as indicated, and all data are expressed as mean±SD. Statistical differences between the control and experimental groups were examined by t-test. P values<0.05 were considered statistically significant.

### 2. Results and Discussion

2.1. GMI does not Exhibit Cytotoxic Effects on HEK293T/ACE2 Cells but Abolishes SARS-CoV-2 Pseudovirus Infection

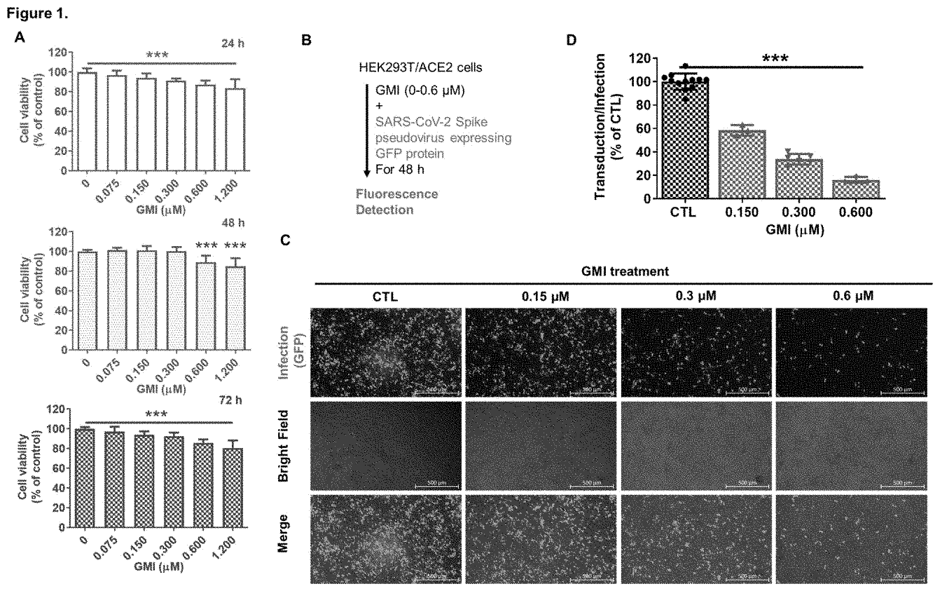

ACE2 is the pivotal receptor for SARS-CoV-2 infection [Reference 22]. To examine the efficacy and potential mechanisms of GMI in the prevention of SARS-CoV-2 infection, we established ACE2 overexpressing HEK293T cells, and noted them as HEK293T/ACE2. Initially, we examined the cytotoxic effects of GMI on HEK293T/ACE2 cells. Using the MTT assay, we found that GMI did not significantly inhibit cell viability of HEK293T/ACE2 in 24 to 72 h of treatment (FIG. 1A). The cytotoxic concentration 50 (CC₅₀) of GMI in the 24, 48 and 72 h treatment was more than 1.2 μM. These results indicated that GMI did not affect cell viability of HEK293T/ACE2 cells.

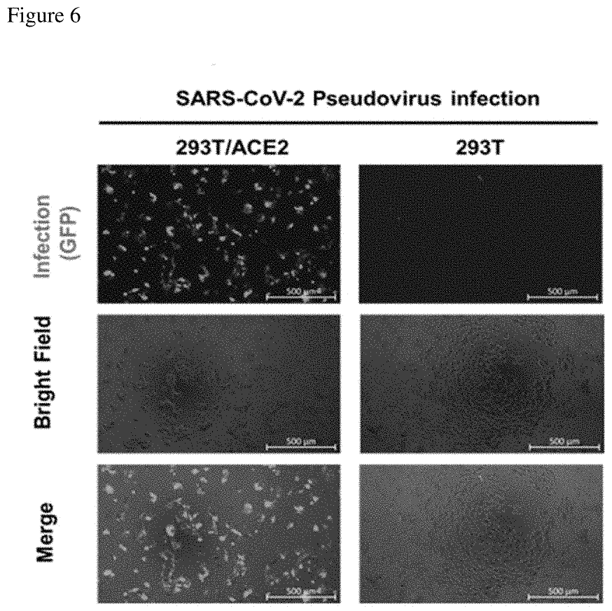

Next, we examined whether GMI could affect SARS-CoV-2 infection. The SARS-CoV-2 Spike pseudovirus expressing GFP protein was applied for evaluating the effects of GMI on SARS-CoV-2 infection. The infectivity of Spike pseudovirus can be analyzed by fluorescence microscopy and flow cytometry. Initially, we demonstrated that SARS-CoV-2 Spike pseudovirus expressing GFP protein effectively infected HEK293T/ACE2 cells but not HEK293T cells (FIG. 6). Next, HEK293T/ACE2 cells were treated with GMI and concomitantly infected with SARS-CoV-2 Spike pseudovirus for 48 h (FIG. 1B). As expected, GMI effectively reduced SARS-CoV-2 pseudovirus infection of HEK293T/ACE2 cells by showing far less GFP-positive cells (FIG. 1C). Simultaneously, we used flow cytometry to quantify the percentage of GFP-positive cells in each sample and found that GMI significantly reduced the transduction rate of Spike pseudovirus to HEK293T/ACE2 cells by up to 80% while using GMI at the concentration of 0.6 μM (FIG. 1D). Together, these findings suggest that GMI may act as a potential agent to inhibit SARS-CoV-2 infection.

2.2. GMI Binds to S2 Domain of Spike Protein and Slightly Reduces Interaction Between ACE2 and Spike

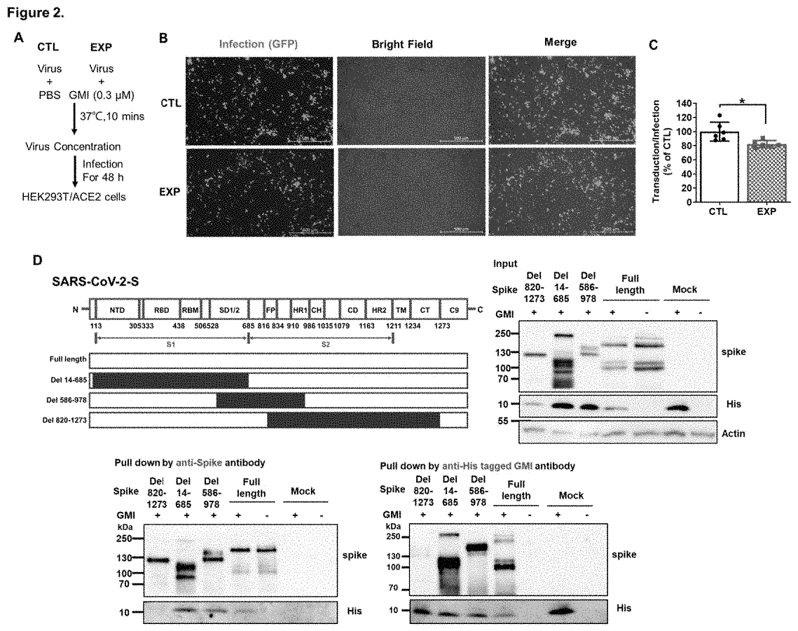

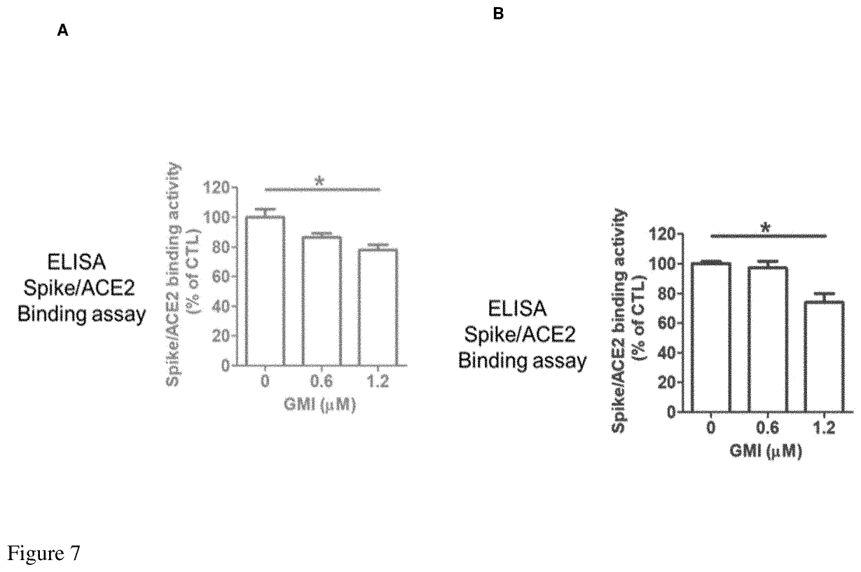

Currently, blocking the interaction between SARS-CoV-2-spike (S) and ACE2 is a potential therapeutic direction in treating SARS-CoV-2 infection [Reference 6]. To validate the role of GMI on virus infection, we initially hypothesized that GMI may block SARS-CoV-2-S/ACE2 interactions. We conducted the ACE2/Spike binding assay and found that SARS-CoV-2-S/ACE2 interactions were slightly reduced by 20% after the incubation of GMI at 1.2 μM with either SARS-CoV-2-S or ACE2 (FIG. 7). In addition, to examine whether GMI could attract the virus and reduce its infection rate, the virus was pretreated with GMI for 10 mins and then harvested by centrifugation (FIG. 2A). Similar to the results of the in vitro binding assay, we found that the pretreatment of GMI on the virus reduced the infection rate by 20% (FIGS. 2B-C). These findings aroused our interest in whether GMI might interact with SARS-CoV-2-S. We next constructed the expression plasmids encoding different SARS-CoV-2-S mutants and overexpressed on the HEK293T cells. By using co-immunoprecipitation assay, we found that GMI could physically interact with the full-length Spike protein and this interaction was compromised by using the Spike protein with deletion at S2 domain (FIG. 2D). It is well-known that receptor-binding domain (RBD) in S1 subunit of SARS-CoV-2 is a key for binding to ACE2 [Reference 5]. Here, we found that GMI interacted with S2 subunit of SARS-CoV-2, suggesting that GMI could not directly affect the interaction between spike and ACE2. Taken together, GMI-inhibited SARS-CoV-2 pseudovirus infection did not fully depend on attacking the virus, but the inhibition might be due to the slight interference in SARS-CoV-2-S/ACE2 interactions via binding to S2 domain of SARS-CoV-2-S or other mechanisms.

2.3. GMI Reduces ACE2 Levels Via the Induction of Protein Degradation in HEK293T/ACE2 Cells

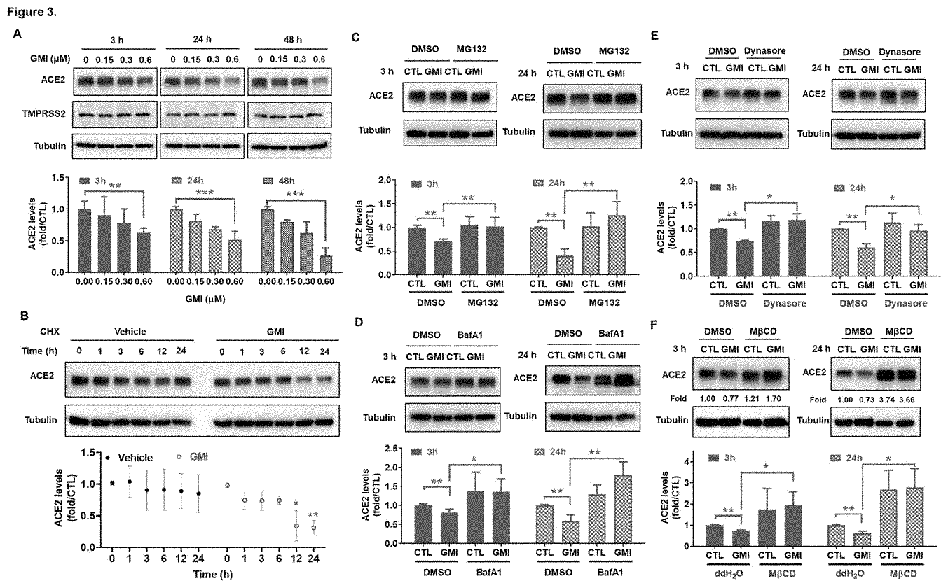

To analyze the potential mechanism of GMI which prevented SARS-CoV2 pseudovirus infection, we investigated whether GMI could directly affect the protein levels of ACE2 and TMPRSS2 on HEK293T/ACE2 cells. As shown in FIG. 3A, we found that GMI reduced expressions of ACE2 in short and long-term treatment; however, GMI did not affect the TMPRSS2 levels. These results suggested that GMI may specifically target ACE2 in HEK293T/ACE2 cells. To further inspect the mechanism of GMI on the regulation of ACE2 expression, we initially examined whether GMI-induced the activation of Adam17, which could cleave the ACE2 [23]. However, GMI did not enhance the activity of Adam17 (data not shown). We thus hypothesized that GMI may interfere with the stability of ACE2. We used cycloheximide (CHX), a ribosome inhibitor, to block the protein synthesis, and found that the co-treatment of GMI and CHX effectively reduced ACE2 levels compared to CHX individual treatment (FIG. 3B). The findings suggested that GMI may induce ACE2 degradation.

Next, we examined the protein degradation pathways in GMI-treated HEK293T/ACE2 cells. Previous studies showed that proteasome contributes to ACE2 degradation [References 24-26]. We therefore investigated whether GMI-induced ACE2 degradation was related to proteasome pathways. Using the proteasome inhibitor, MG132, we found that MG132 could abolish GMI-reduced ACE2 levels in short and long-term treatments (FIG. 3C). In addition, evidence shows that angiotensin-II reduces ACE2 by stimulation of the lysosomal degradation pathway [Reference 27]. Using the lysosome inhibitors, BafA1, to block the lysosome activity, we also found that ACE2 levels were maintained in HEK293T/ACE2 cells which were treated with GMI (FIG. 3D). These results indicated that GMI may disturb the stability of ACE2, which could lead to ACE2 degradation. Herein, we found that GMI could induce ACE2 degradation via the proteasome or lysosome pathway. Evidence shows that membrane proteins are usually degraded due to ubiquitination-dependent endocytosis [Reference 28, 29]. This finding shows that GMI may activate two protein degradation systems, causing endocytosis and degradation of ACE2.

Increasing evidence shows that coronaviruses enter host cells via binding to ACE2 and triggering clathrin or lipid raft-dependent endocytosis [Reference 30]. We thereby examined the endocytosis pathways in GMI-induced ACE2 degradation. Initially, dynasore, a dynamin inhibitor, was chosen to inhibit clathrin-dependent endocytosis [Reference 31]. As shown in FIG. 3E, we found that dynasore increased ACE2 levels and abolished GMI-reduced ACE2 expression. Next, we used the methyl-β-cyclodextrin (MβCD) to disrupt lipid raft. We found that MβCD dramatically increased ACE2 levels for the long-term treatment (FIG. 3F). Specifically, MβCD rescued GMI-induced ACE2 levels (FIG. 3F). We also examined the interaction between GMI and ACE2. However, we did not identify the complex of ACE2/GMI by using the immunoprecipitation assay (data not shown). Together, these results suggest that GMI downregulated expression of ACE2 via two steps: 1. GMI-induced clathrin and lipid raft-dependent endocytosis of ACE2; 2. ACE2 was then degraded via lysosomal and proteasomal pathways.

2.4. Discontinuous GMI Exposure Maintains the Inhibition Rate of SARS-CoV-2 Pseudovirus Infection

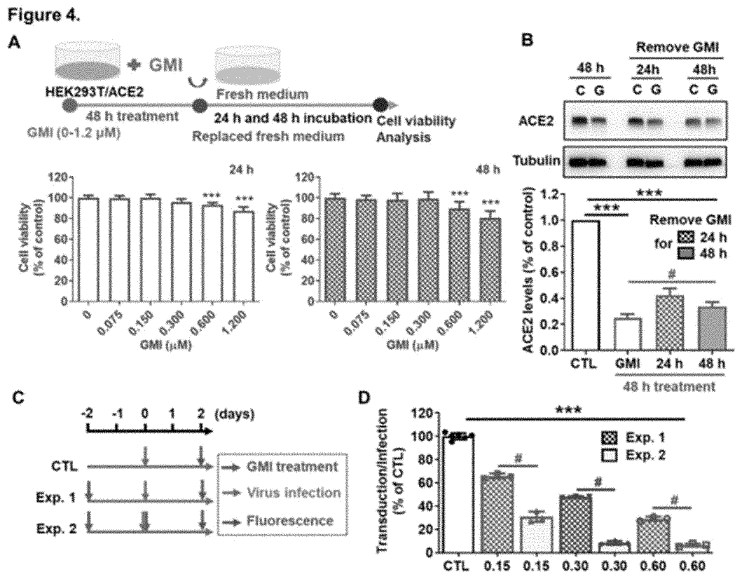

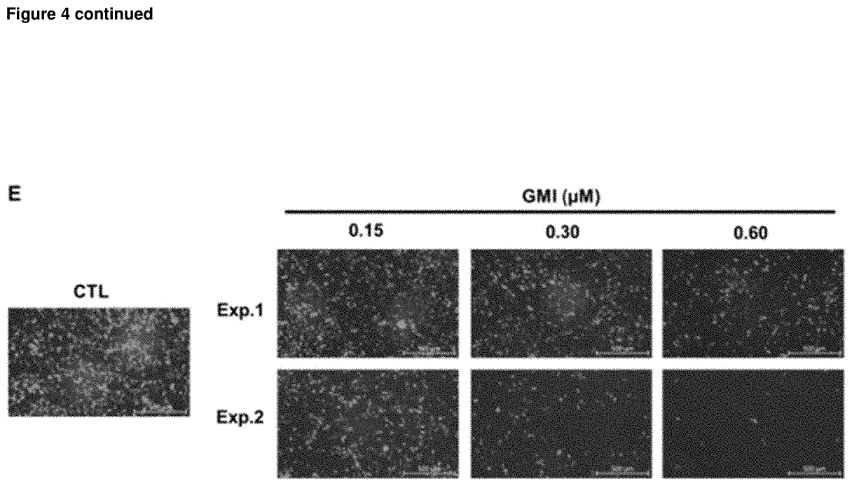

To analyze the efficacy of GMI on regulating ACE2 levels and inhibition rate of SARS-CoV-2 pseudovirus infection, we conducted the discontinuous GMI exposure experiments and examined the cell viability of HEK293T/ACE2 cells. HEK293T/ACE2 cells were exposed to GMI for 48 h and the cell viability was examined after removing GMI. As shown in FIG. 4A, we found that when GMI was given for 48 h, removing GMI (1.2 μM) from the HEK293T/ACE2 cells slightly inhibited the cell viability by approximately 10%. Therefore, we further examined the treatment condition on ACE2 levels. As expected, GMI (0.6 μM) dramatically reduced ACE2 levels (FIG. 4B). Specifically, GMI substantially downregulated ACE2 levels by 80% after 48 h treatment. Interestingly, if GMI was removed after 48 h treatment, ACE2 would slightly rise after another 24 and 48 h (FIG. 4B). However, the ACE2 levels still cannot return to 100% after GMI removal. These results indicate that after GMI was removed from the host cells, ACE2 levels did not quickly recover to normal levels, suggesting that GMI treatment can prolong the efficacy of protection against SARS-CoV-2 infection. Therefore, we conducted experiments to examine the efficacy of GMI on infection after removing GMI (FIG. 4C). After 48 h of GMI treatment, GMI was removed, and at the same time, the SARS-CoV-2 pseudovirus was given to the HEK293T/ACE2 cells for 48 h. We found that GMI effectively inhibited the virus infection in a concentration-dependent manner (Exp. 1 vs CTL; FIGS. 4D-E). Moreover, with the continuous treatment of GMI SARS-CoV-2 pseudovirus infection, the inhibition rate of infection increased to more than 80%. Specifically, the continuous treatment of GMI significantly reduced SARS-CoV-2 pseudovirus infection compared to discontinuous administration (Exp. 2 vs Exp. 1; FIGS. 4D-E). Taken together, GMI exhibits great efficacy with respect to the inhibition of SARS-CoV-2 pseudovirus infection.

2.5. GMI does not Exhibit a Cytotoxic Effect but Reduces ACE2 Expression in Lung WI38-2RA Cells

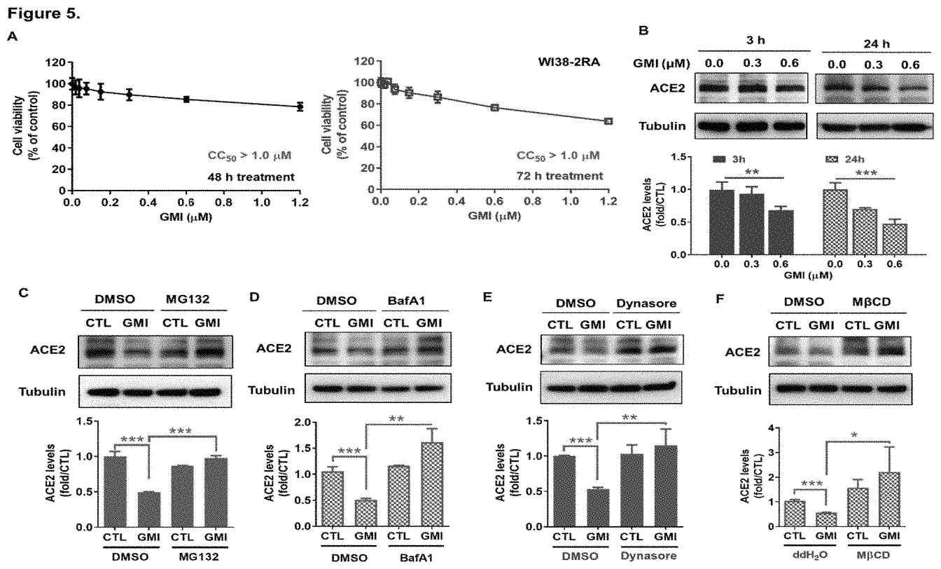

As shown in the above findings, we found that GMI effectively downregulated ACE2 expression via induction of the protein degradation system. Therefore, we examined the effects of GMI on normal human lung cells by inspecting the cytotoxic effects of GMI on lung WI38-2RA cells. Using the MTT assay, we found that GMI did not significantly inhibit cell viability of WI38-2RA after 48 and 72 h treatment (FIG. 5A). The CC₅₀ of GMI in the 48 and 72 h treatment were more than 1.0 μM. Moreover, we found that GMI did not affect the cell viability of lung fibroblast MRC-5 and alveolar macrophage MH-S (data not shown). Together, GMI did not affect cell viability of normal lung cells.

Next, we investigated whether GMI could affect the protein levels of ACE2 in WI38-2RA cells. As shown in FIG. 5B, the data showed that GMI reduced expressions of ACE2 in short and long-term treatments; specifically, GMI dramatically downregulated ACE2 after 24 h treatment. These results suggested that GMI may specifically target ACE2 in WI38-2RA cells. Similarly, to analyze the mechanism of GMI on regulation of ACE2 expression, we examined whether GMI-induced ACE2 degradation via the degradation system. As shown in FIGS. 5C-D, either MG132 or BafA1 effectively rescued GMI-reduced ACE2 levels. Moreover, dynasore and MβCD rescued GMI-induced ACE2 levels (FIGS. 5E-F). Together, these results indicated that GMI may potentially reduce SARS-CoV-2 infection via the induction of ACE2 degradation.

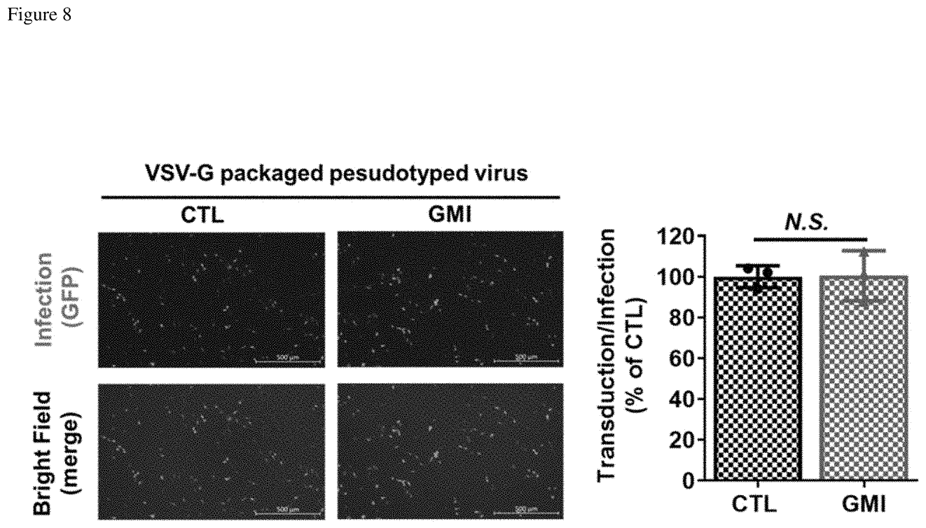

In this study, we attempted to analyze the anti-COVID-19 function of GMI. In addition, we found that GMI did not inhibit VSV-G pseudotyped virus infection (FIG. 8), suggesting that GMI may specifically target SARS-CoV-2 infection. Currently, multiple potential anti-SARS-CoV-2 strategies such as receptor binding, entry/fusion, and replication have been analyzed [Reference 32]. Unlike the small compound to target replication-related enzymes, GMI is a fungal protein which may interfere with the interaction between SARS-CoV-2 and host cells. We therefore examined whether GMI blocked the binding of ACE2 to SARS-CoV-2-S. However, GMI slightly abolished the SARS-CoV-2-S/ACE2 interactions by 20%, suggesting that GMI may interact with SARS-CoV-2-S or ACE2. We found that GMI did not bind to ACE2 (data not shown). In contrast, GMI could interact with S2 subunit of SARS-CoV-2-S. Previous studies showed that a carbohydrate-binding protein (noted FRIL) from Lablab purpureus effectively blocks the infections of SARS-CoV-2 via binding to N-glycan of glycoprotein [Reference 33]. Specifically, FRIL interacting with the virus to form aggregates may prevent virus entry or trap the virus in the late endosome. Whether GMI exhibits functions similar to those of FRIL needs to be explored in the future. In parallel, targeting the receptors of SARS-CoV-2-S on host cells may be an intervention strategy [Reference 34]. ACE2 is a well-known membrane protein that controls SARS-CoV-2 infection [Reference 34]. We found that GMI effectively reduced ACE2 levels on the host cells. Specifically, GMI triggered the endocytosis and degradation of ACE2 in host cells, resulting in reducing the SARS-CoV-2 pseudovirus infection. Until now, few studies have focused on targeting ACE2 degradation as a novel strategy for preventing the SARS-CoV-2 infection [Reference 11]. Herein, we provide a novel fungal protein, GMI, which reduced SARS-CoV-2 pseudovirus infection in vitro. GMI will need to be investigated in animal models to test its efficacy regarding the infection and ACE2 levels on lung tissue.

### REFERENCES

[1] T. Kustin, N. Harel, U. Finkel, S. Perchik, S. Harari, M. Tahor, I. Caspi, R. Levy, M. Leshchinsky, S. Ken Dror, G. Bergerzon, H. Gadban, F. Gadban, E. Eliassian, O. Shimron, L. Saleh, H. Ben-Zvi, E. Keren Taraday, D. Amichay, A. Ben-Dor, D. Sagas, M. Strauss, Y. Shemer Avni, A. Huppert, E. Kepten, R. D. Balicer, D. Netzer, S. Ben-Shachar, A. Stem, Evidence for increased breakthrough rates of SARS-CoV-2 variants of concern in BNT162b2-mRNA-vaccinated individuals, Nat Med 27(8) (2021) 1379-1384.
[2] J. Abbasi, COVID-19 mRNA Vaccines Blunt Breakthrough Infection Severity, JAMA 326(6) (2021) 473.
[3] D. Planas, D. Veyer, A. Baidaliuk, I. Staropoli, F. Guivel-Benhassine, M. M. Rajah, C. Planchais, F. Porrot, N. Robillard, J. Puech, M. Prot, F. Gallais, P. Gantner, A. Velay, J. Le Guen, N. Kassis-Chikhani, D. Edriss, L. Belec, A. Seve, L. Courtellemont, H. Pere, L. Hocqueloux, S. Fafi-Kremer, T. Prazuck, H. Mouquet, T. Bruel, E. Simon-Loriere, F. A. Rey, O. Schwartz, Reduced sensitivity of SARS-CoV-2 variant Delta to antibody neutralization, Nature 596(7871) (2021) 276-280.
[4] J. Yang, S. J. L. Petitjean, M. Koehler, Q. Zhang, A. C. Dumitru, W. Chen, S. Derclaye, S. P. Vincent, P. Soumillion, D. Alsteens, Molecular interaction and inhibition of SARS-CoV-2 binding to the ACE2 receptor, Nat Commun 11(1) (2020) 4541.
[5] J. Lan, J. Ge, J. Yu, S. Shan, H. Zhou, S. Fan, Q. Zhang, X. Shi, Q. Wang, L. Zhang, X. Wang, Structure of the SARS-CoV-2 spike receptor-binding domain bound to the ACE2 receptor, Nature 581(7807) (2020) 215-220.
[6] N. Zamorano Cuervo, N. Grandvaux, ACE2: Evidence of role as entry receptor for SARS-CoV-2 and implications in comorbidities, Elife 9 (2020).
[7] A. Glasgow, J. Glasgow, D. Limonta, P. Solomon, I. Lui, Y. Zhang, M. A. Nix, N. J. Rettko, S. Zha, R. Yamin, K. Kao, O. S. Rosenberg, J. V. Ravetch, A. P. Wiita, K. K. Leung, S. A. Lim, X. X. Zhou, T. C. Hobman, T. Kortemme, J. A. Wells, Engineered ACE2 receptor traps potently neutralize SARS-CoV-2, Proc Natl Acad Sci USA 117(45) (2020) 28046-28055.
[8] T. M. Abd El-Aziz, A. Al-Sabi, J. D. Stockand, Human recombinant soluble ACE2 (hrsACE2) shows promise for treating severe COVID-19, Signal Transduct Target Ther 5(1) (2020) 258.
[9] A. Zoufaly, M. Poglitsch, J. H. Aberle, W. Hoepler, T. Seitz, M. Traugott, A. Grieb, E. Pawelka, H. Laferl, C. Wenisch, S. Neuhold, D. Haider, K. Stiasny, A. Bergthaler, E. Puchhammer-Stoeckl, A. Mirazimi, N. Montserrat, H. Zhang, A. S. Slutsky, J. M. Penninger, Human recombinant soluble ACE2 in severe COVID-19, Lancet Respir Med 8(11) (2020) 1154-1158.
[10] J. Ge, R. Wang, B. Ju, Q. Zhang, J. Sun, P. Chen, S. Zhang, Y. Tian, S. Shan, L. Cheng, B. Zhou, S. Song, J. Zhao, H. Wang, X. Shi, Q. Ding, L. Liu, J. Zhao, Z. Zhang, X. Wang, L. Zhang, Antibody neutralization of SARS-CoV-2 through ACE2 receptor mimicry, Nat Commun 12(1) (2021) 250.
[11] S. Su, J. Chen, Y. Wang, L. M. Wong, Z. Zhu, G. Jiang, P. Liu, Lenalidomide downregulates ACE2 protein abundance to alleviate infection by SARS-CoV-2 spike protein conditioned pseudoviruses, Signal Transduct Target Ther 6(1) (2021) 182.
[12] C.-H. Lin, G.-T. Sheu, Y.-W. Lin, C.-S. Yeh, Y.-H. Huang, Y.-C. Lai, J.-G. Chang, J.-L. Ko, A new immunomodulatory protein from Ganoderma microsporum inhibits epidermal growth factor mediated migration and invasion in A549 lung cancer cells, Process Biochemistry 45(9) (2010) 1537-1542.
[13] I. L. Hsin, C. C. Ou, M. F. Wu, M. S. Jan, Y. M. Hsiao, C. H. Lin, J. L. Ko, GMI, an Immunomodulatory Protein from Ganoderma microsporum, Potentiates Cisplatin-Induced Apoptosis via Autophagy in Lung Cancer Cells, Mol Pharm 12(5) (2015) 1534-43.
[14] I. L. Hsin, G. T. Sheu, M. S. Jan, H. L. Sun, T. C. Wu, L. Y. Chiu, K. H. Lue, J. L. Ko, Inhibition of lysosome degradation on autophagosome formation and responses to GMI, an immunomodulatory protein from Ganoderma microsporum, Br J Pharmacol 167(6) (2012) 1287-300.
[15] I. L. Hsin, C. C. Ou, T. C. Wu, M. S. Jan, M. F. Wu, L. Y. Chiu, K. H. Lue, J. L. Ko, GMI, an immunomodulatory protein from Ganoderma microsporum, induces autophagy in non-small cell lung cancer cells, Autophagy 7(8) (2011) 873-82.
[16] I. L. Hsin, J. C. Hsu, W. J. Wu, H. J. Lu, M. F. Wu, J. L. Ko, GMI, a fungal immunomodulatory protein from Ganoderma microsporum, induces apoptosis via beta-catenin suppression in lung cancer cells, Environ Toxicol 33(9) (2018) 955-961.
[17] I. L. Hsin, L. Y. Chiu, C. C. Ou, W. J. Wu, G. T. Sheu, J. L. Ko, CD133 inhibition via autophagic degradation in pemetrexed-resistant lung cancer cells by GMI, a fungal immunomodulatory protein from Ganoderma microsporum, Br J Cancer (2020).
[18] W. Jiang, R. Hua, M. Wei, C. Li, Z. Qiu, X. Yang, C. Zhang, An optimized method for high-titer lentivirus preparations without ultracentrifugation, Scientific Reports 5(1) (2015) 13875.
[19] T. Y. Lin, W. J. Hua, H. Yeh, A. J. Tseng, Functional proteomic analysis reveals that fungal immunomodulatory protein reduced expressions of heat shock proteins, which correlates to apoptosis in lung cancer cells, Phytomedicine 80 (2021) 153384.
[20] W. L. Qiu, A. J. Tseng, H. Y. Hsu, W. H. Hsu, Z. H. Lin, W. J. Hua, T. Y. Lin, Fucoidan increased the sensitivity to gefitinib in lung cancer cells, which correlates to a reduction of TGFbeta-mediated Slug expression, Int J Biol Macromol 153 (2020) 796-805.
[21] T. Y. Lin, H. Y. Hsu, Ling Zhi-8 reduces lung cancer mobility and metastasis through disruption of focal adhesion and induction of MDM2-mediated Slug degradation, Cancer Lett 375(2) (2016) 340-8.
[22] H. Jia, E. Neptune, H. Cui, Targeting ACE2 for COVID-19 Therapy: Opportunities and Challenges, Am J Respir Cell Mol Biol 64(4) (2021) 416-425.
[23] A. Heurich, H. Hofmann-Winkler, S. Gierer, T. Liepold, O. Jahn, S. Pöhlmann, TMPRSS2 and ADAM17 cleave ACE2 differentially and only proteolysis by TMPRSS2 augments entry driven by the severe acute respiratory syndrome coronavirus spike protein, J Virol 88(2) (2014) 1293-1307.
[24] H. Shen, J. Zhang, C. Wang, P. P. Jain, M. Xiong, X. Shi, Y. Lei, S. Chen, Q. Yin, P. A. Thistlethwaite, J. Wang, K. Gong, Z. Y. Yuan, J. X. Yuan, J. Y. Shyy, MDM2-Mediated Ubiquitination of Angiotensin-Converting Enzyme 2 Contributes to the Development of Pulmonary Arterial Hypertension, Circulation 142(12) (2020) 1190-1204.
[25] X. Liu, N. Yang, J. Tang, S. Liu, D. Luo, Q. Duan, X. Wang, Downregulation of angiotensin-converting enzyme 2 by the neuraminidase protein of influenza A (H1N1) virus, Virus Res 185 (2014) 64-71.
[26] A. Abdul-Hafez, J. Markey, B. Uhal, EVIDENCE FOR ACE-2 UBIQUITINATION AND PROTEASOMAL PROCESSING IN LUNG EPITHELIUM, The FASEB Journal 31(S1) (2017) 725.5-725.5.
[27] M. R. Deshotels, H. Xia, S. Sriramula, E. Lazartigues, C. M. Filipeanu, Angiotensin II Mediates Angiotensin Converting Enzyme Type 2 Internalization and Degradation Through an Angiotensin II Type I Receptor& #x2013; Dependent Mechanism, Hypertension 64(6) (2014) 1368-1375.
[28] M. D. Marmor, Y. Yarden, Role of protein ubiquitylation in regulating endocytosis of receptor tyrosine kinases, Oncogene 23(11) (2004) 2057-70.
[29] J. S. Weinberg, D. G. Drubin, Regulation of clathrin-mediated endocytosis by dynamic ubiquitination and deubiquitination, Curr Biol 24(9) (2014) 951-9.
[30] J. Bian, Z. Li, Angiotensin-converting enzyme 2 (ACE2): SARS-CoV-2 receptor and RAS modulator, Acta Pharm Sin B 11(1) (2021) 1-12.
[31] T. Kirchhausen, E. Macia, H. E. Pelish, Use of dynasore, the small molecule inhibitor of dynamin, in the regulation of endocytosis, Methods Enzymol 438 (2008) 77-93.
[32] B. Hu, H. Guo, P. Zhou, Z.-L. Shi, Characteristics of SARS-CoV-2 and COVID-19, Nature Reviews Microbiology 19(3) (2021) 141-154.
[33] Y. M. Liu, M. Shahed-Al-Mahmud, X. Chen, T. H. Chen, K. S. Liao, J. M. Lo, Y. M. Wu, M. C. Ho, C. Y. Wu, C. H. Wong, J. T. Jan, C. Ma, A Carbohydrate-Binding Protein from the Edible Lablab Beans Effectively Blocks the Infections of Influenza Viruses and SARS-CoV-2, Cell Rep 32(6) (2020) 108016.
[34] J. Shang, G. Ye, K. Shi, Y. Wan, C. Luo, H. Aihara, Q. Geng, A. Auerbach, F. Li, Structural basis of receptor recognition by SARS-CoV-2, Nature 581(7807) (2020) 221-224.

What is claimed is:

1. A method for preventing, inhibiting, reducing and/or eliminating CoV infection and/or likelihood of CoV infection in a subject, comprising administering an effective amount of Ganoderma immunomodulatory protein, a recombinant thereof or a fragment thereof to the subject.

2. The method of claim 1, wherein the Ganoderma immunomodulatory protein, a recombinant thereof or a fragment thereof is derived from Ganoderma lucidum, Ganoderma tsugae, Ganoderma microsporum or Ganoderma sinensis.

3. The method of claim 1, wherein the Ganoderma immunomodulatory protein, a recombinant thereof or a fragment thereof is derived from Ganoderma microsporum.

4. The method of claim 1, wherein the Ganoderma immunomodulatory protein or a recombinant thereof or a fragment thereof comprises an amino acid sequence of SEQ ID NO: 3.

5. The method of claim 1, wherein the recombinant of Ganoderma immunomodulatory protein comprises an amino acid sequence of SEQ ID NO: 4.

6. The method of claim 1, wherein the fragment of Ganoderma immunomodulatory protein comprises an amino acid sequence selected from the group consisting of SEQ ID NOs: 1 to 2.

7. The method of claim 1, wherein the CoV is alpha-CoV, beta-CoV, gamma-CoV, and delta-CoV2.

8. The method of claim 1, wherein the Ganoderma immunomodulatory protein or a recombinant thereof or a fragment thereof induces ACE2 degradation.

9. The method of claim 8, wherein the ACE2 degradation is induced via activating the protein degradation system, including proteasome and lysosome.

10. The method of claim 1, wherein the subject is vaccinated.

11. The method of claim 10, wherein the subject is vaccinated with a COVID-19 coronavirus mRNA vaccine, COVID-19 coronavirus spike protein vaccine or COVID-19 coronavirus viral vector vaccine.

12. The method of claim 1, wherein the subject is treating or has been treated with a therapeutic agent against CoV.

13. The method of claim 1, wherein the subject is administered one or more further therapeutic agents against CoV before or after or at the same time the Ganoderma immunomodulatory protein or a recombinant thereof or a fragment is administered.

14. The method of claim 1, wherein the Ganoderma immunomodulatory protein or a recombinant thereof or a fragment can be administered with one or more further therapeutic agents against CoV concurrently, sequentially or separately.

15. The method of claim 1, wherein the Ganoderma immunomodulatory protein or a recombinant thereof or a fragment thereof is administered orally, parenterally, topically or by inhalation.

16. The method of claim 1, wherein the Ganoderma immunomodulatory protein or a recombinant thereof or a fragment thereof is administered by inhaler to the respiratory tract.
